(19) United States
(12) Patent Application Publication
(10) Pub. No.: US 2017/0035540 A1
(43) Pub. Date: Feb. 9, 2017

(54) CLEANING, DRYING, AND DISPENSING APPLICATOR PAD

(71) Applicant: Centrix, Inc., Shelton, CT (US)

(72) Inventor: Pengguang YU, Branford, CT (US)

(21) Appl. No.: 15/334,184

(22) Filed: Oct. 25, 2016

Related U.S. Application Data
(63) Continuation of application No. PCT/US2015/028898, filed on May 1, 2015.
(60) Provisional application No. 61/988,361, filed on May 5, 2014.

(51) Int. Cl. A61C 19/06; A61Q 11/00; A61K 8/02; A61K 8/92; A61K 8/21

(52) U.S. Cl. CPC A61C 19/066; A61K 8/92; A61K 8/21; A61K 8/0208; A61Q 11/00; A61K 2800/87

(57) ABSTRACT

An applicator pad for cleaning and drying a surface prior to applying a material. The applicator pad cleans and dries a tooth prior to application of a wax material. The wax material may comprise a tooth whitening agent or a fluoride material. In one embodiment a plurality of flexible absorbent posts extend above the wax material surface. In another embodiment a grid extends above the wax material surface. In another embodiment two different wax material portions are divided by a fold line. In another embodiment a cleaning and drying portion is placed adjacent the wax material applying portion. Cleaning and drying of the tooth surface prior to the application of the wax material facilitates the adherence of the wax material onto the tooth surface. The applicator pad is particularly beneficial for tooth whitening and maintenance.

### RELATED APPLICATIONS

This application is a continuation of International Application PCT/US2015/028898, with an international filing date of May 1, 2015, which claims the benefit of U.S. Provisional Application No. 61/988,361 filed May 5, 2014, all of which are herein incorporated by reference.

### FIELD OF THE INVENTION

The present invention relates in general to applying a material to a moist surface, and particularly to applying a protectant or treatment material to a surface of a tooth.

### BACKGROUND OF THE INVENTION

Often a dental material must be applied to the surface of a tooth and held in position for a desired period of time. It is often difficult to apply a material to a wet or moist surface. This is especially problematic when applying a desired material, particularly hydrophobic materials, to a tooth surface in the mouth. Often the surface of the tooth is coated resulting in a lack of adhesion of the applied material. Therefore, it is difficult to apply a wax or protective material to the surface of the tooth so that it will remain on the tooth surface for a required or desired period of time.

There have been attempts to provide a delivery system to apply a material to a tooth surface, such as a protectant or tooth whitening substance. One such material uses a protectant is disclosed in U.S. Pat. No. 5,888,480 entitled “Methods, Compositions, and Dental Delivery Systems for the Protection of the Services of Teeth” issuing to Homola et al. on Mar. 30, 1999, which is herein incorporated by reference. Therein disclosed is a barrier layer utilizing electrostatic charge of distribution to facilitate adherence of a material to a tooth surface. Another delivery system adapted for providing a tooth whitening substance is disclosed in U.S. Pat. No. 5,879,691 entitled “Delivery System for a Tooth Whitener Using a Strip of Material Having a Low Flexural Stiffness” issuing to Sagel et al. on Mar. 9, 1999, which is herein incorporated by reference. Therein disclosed is a delivery system for a tooth whitening substance including a strip of flexible material with a tooth whitening substance applied to the strip of material. The substance has adhesive attachment between the strip of material and the surface to hold the delivery system in place for a sufficient time to allow the active to act upon the surface.

While these delivery systems have advanced the art they are not without their difficulties and complexities. The tooth whitener using a strip of material disclosed in U.S. Pat. No. 5,879,691 is inconvenient in that it requires the strip to remain on the tooth to allow the active to act upon the surface which may be inconvenient and is cosmetically awkward.

Accordingly, there is a need for an easy and convenient device for applying a material to the surface of a tooth that will dry and clean the tooth surface prior to applying the material, and in particular a tooth whitening material or agent.

### BRIEF DESCRIPTION OF THE DRAWINGS

FIG. 1 is a plan view schematically illustrating the invention.

FIG. 2 is a cross section taken along line 2-2 in FIG. 1.

FIG. 3 is a cross section taken along line 3-3 in FIG. 2.

FIG. 4 is a perspective view illustrating the assembly of the invention.

FIG. 5A is a perspective view schematically illustrating another embodiment of the invention having absorbent posts or spacers.

FIG. 5B is a perspective view schematically illustrating the embodiment in FIG. 5A showing the flexibility of the absorbent posts or spacers.

FIG. 5C is a cross section schematically illustrating an embodiment with the absorbent posts or spacers having a rounded distal end.

FIG. 6A schematically illustrates another embodiment of the invention having an absorbent grid.

FIG. 6B is a cross section illustrating the structure of the embodiment illustrated in FIG. 6A.

FIG. 6C schematically illustrates the use of the embodiment of the invention illustrated in FIGS. 6A-B on a user's teeth.

FIG. 7 schematically illustrates another embodiment of the invention having an absorbent geometric grid.

FIG. 8A is a perspective view schematically illustrating another embodiment of the invention having a material coating on the transmissible cover.

FIG. 8B is a cross section of the embodiment illustrated in FIG. 8A.

FIG. 9A schematically illustrates another embodiment of the invention having a barrier film with holes.

FIG. 9B is a cross section of the embodiment illustrated in FIG. 9A.

FIG. 10 schematically illustrates another embodiment of the invention having a water repelling grid.

FIG. 11 schematically illustrates another embodiment of the invention having a material impregnated top layer.

FIG. 12A is a cross section schematically illustrating another embodiment of the invention having a plurality of material portions or chambers and a fold line.

FIG. 12B is a plan view of another embodiment of the invention having two separated rectangular shaped material portions.

FIG. 12C is a plan view of another embodiment of the invention having curved or rounded anatomically shaped material portions.

FIG. 12D is a cross section schematically illustrating the embodiment of the invention illustrated in FIG. 12A in use applying a material to the front and back of a tooth surface.

FIG. 13 schematically illustrates a structure of an embodiment of the invention having a medicinal or medicated carrier layer.

FIG. 14A is a perspective view illustrating another embodiment of the invention having a separate drying portion and a separate material applicator portion.

FIG. 14B is a cross section schematically illustrating the embodiment of the invention illustrated in FIG. 14A.

### SUMMARY OF THE INVENTION

The present invention provides an applicator that cleans and dries a surface prior to the application of a material. A material to be applied to a surface, such as a moist surface of a tooth in a person's mouth, is contained between a flexible substrate and a transmissible cover. The transmissible cover permits the material to pass through when pressure is applied to the flexible substrate. The transmissible cover cleans and dries the surface prior to application of the material. In one embodiment flexible posts are formed on the flexible substrate. The flexible posts have a distal end above the material interspersed between the flexible posts. The distal end of the flexible post clean and dry the surface and bend when pressure is applied to the flexible substrate applying the material interspersed therebetween to a surface. In another embodiment a grid is formed on the flexible substrate and the material is interspersed within the grid. Another embodiment has a barrier film having holes therein placed between the transmissible cover and the material providing improved control of the dispensing of the material through the transmissible cover. In another embodiment the material is separated by a fold line forming an outer tooth pad and an inner tooth pad. This embodiment is applicable to simultaneously cleaning, drying, and applying material on the inside surface and the outside surface of a tooth. In yet another embodiment a combination applicator pad is divided into a drying portion and a material applicator portion.

It is an object of the present invention to clean and dry a surface while applying a material.

It is another object of the present invention to placing material on a moist surface with improved adhesion of the material to the surface.

It is an advantage of the present invention that a transmissible cover cleans and dries the surface prior to the application of the material.

It is an advantage of the present invention that it is a storage device for the material which is applied over teeth.

It is another advantage of the present invention that the applicator is not required to remain on the surface after application of the material.

It is yet another advantage of the present invention that cleaning, drying, and applying of the material is accomplished easily and quickly with a single device.

It is a feature of the present invention that a transmissible cover separates a material to be applied from the surface the material is being applied to.

It is another feature of the present invention that the transmissible cover or a structure placed on the transmissible cover cleans and dries the surface of a tooth prior to the application of a material.

It is a feature of the present invention that the material applied may be a tooth whitening agent.

These and other objects, advantages, and features will become readily apparent in view the following detailed description.

### DETAILED DESCRIPTION OF THE INVENTION

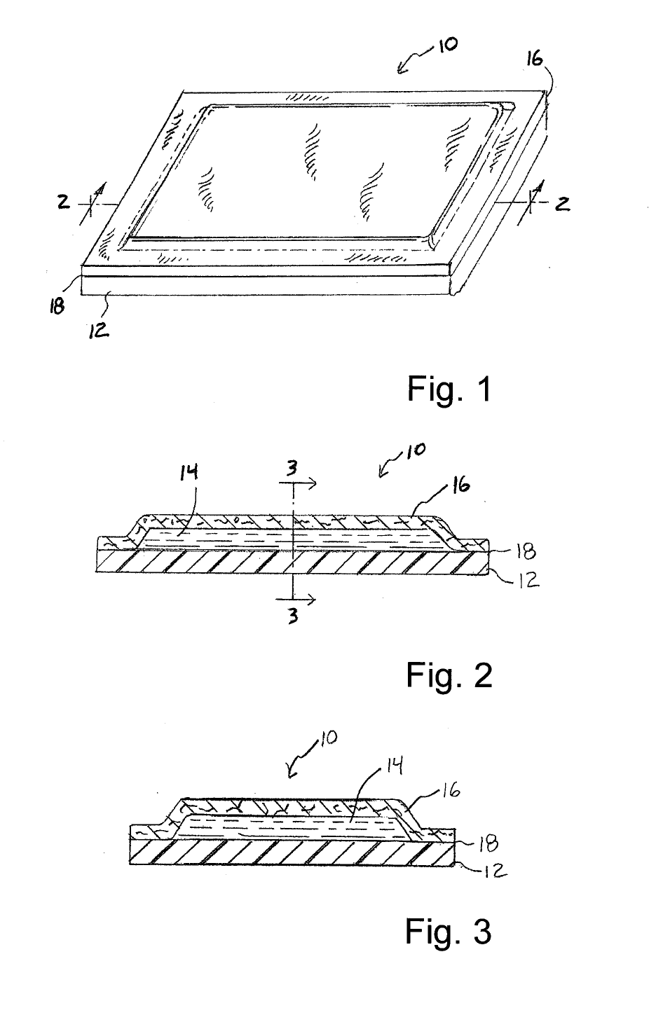

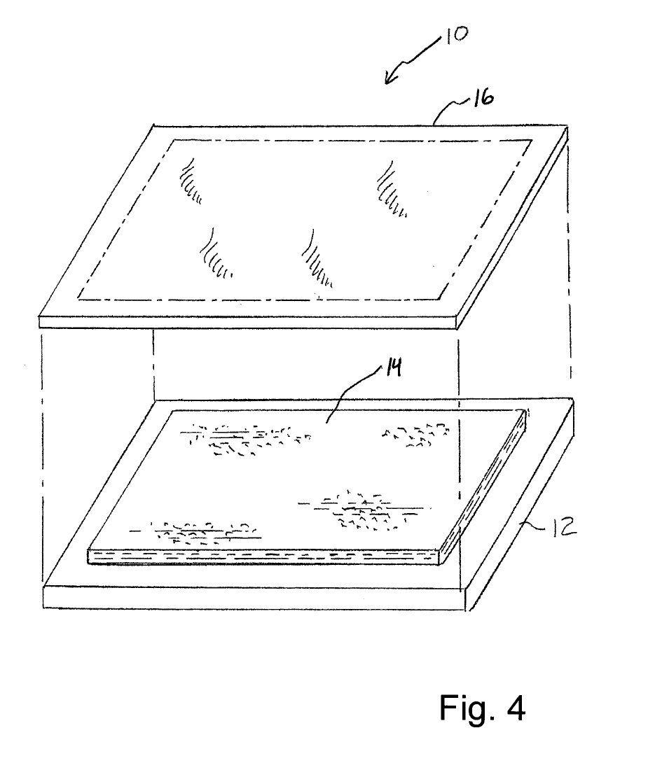

FIGS. 1-4 schematically illustrates an embodiment of the present invention for applying a wax, gel, liquid, or other material to the surface of a tooth or other moist surface. The cleaning, drying, and dispensing applicator pad 10 has an impervious flexible substrate 12 with a material 14 placed thereon. Material 14 may be a wax, gel, cream, liquid and other material to be applied to a moist or coated tooth or other surface. The material 14 may be a wax like material that is used to protect the tooth to prevent staining or discoloration so as to maintain and protect a tooth that has been whitened. Use of the invention will effectively eliminate the need for the frequently whitening of teeth. The present invention may also contain a tooth whitening agent, fluoride varnish material, fluoride material, or other material used in protecting or treating a tooth. The material 14 is covered with a transmissible cover 16. A seal 18 is formed around the perimeter for holding the material 14 within the applicator pad 10. The transmissible cover 16 may be made of any porous substance that can transmit the material 14 there through. The transmissible cover 16 may be made of a natural, cotton, or synthetic, plastic, fabric, machined sponges with holes, or similar like material. The top layer may also be treated to kill germs.

In operation, the transmissible cover 16 is wiped or tapped across a tooth surface wiping it clean and absorbing any moisture. Subsequently or simultaneously, the applicator pad 10 is gently pressed pushing the material 14 through the transmissible cover 16 applying the material 14 to the cleaned surface of the tooth. Accordingly, the material 14 being applied to a clean surface of the tooth results in the material 14 forming a coating that adheres for an extended period of time to the tooth surface. The extended period of adhesion of the coating of material 14 on the tooth surface results in a tooth surface being protected or being treated for a longer period of time. Additionally, if a whitening agent is used, the whitening agent will adhere to the tooth surface providing longer period of time to be active without the need of the applicator to remain on the tooth surface. The whitening of teeth can actively occur without any visible indication or discomfort to the user.

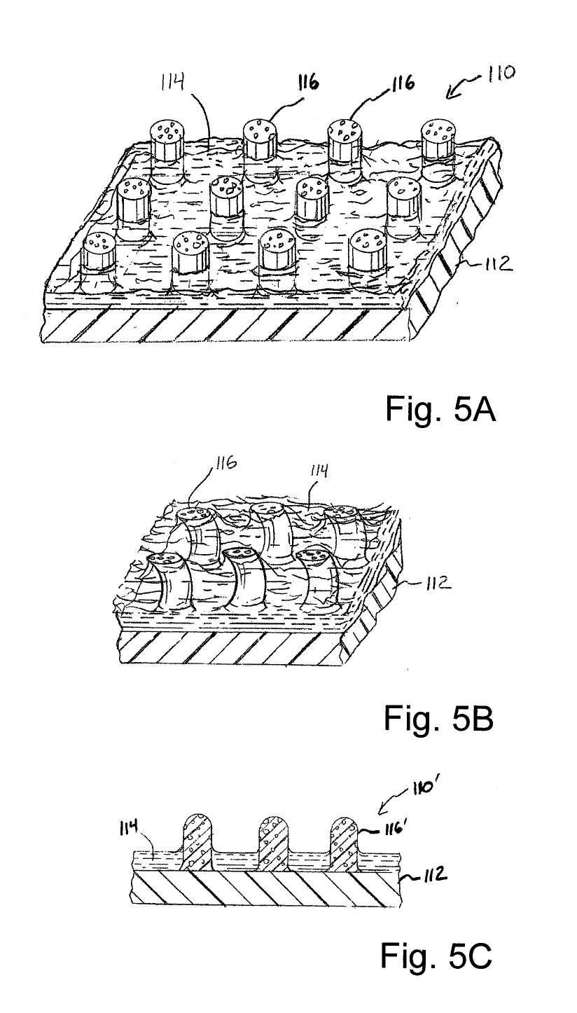

FIGS. 5A-B schematically illustrate another embodiment of the present invention. In this embodiment the cleaning, drying, and dispensing applicator pad 110 comprises a substrate 112 having a material 114 thereon. Absorbent posts 116 extend from the substrate 112 and have the material 114 dispersed there between. The absorbent posts 116 are flexible and when pressure is applied will slightly bend so that the material 114 is in contact with the tooth surface and is therefore applied to the surface of a tooth. The top surfaces of the absorbent posts 116 may be flat so as to effectively clean and absorb any moisture or coating on the tooth. The absorbent posts 116 are made of an absorbent material that may be either natural or synthetic. The absorbent posts 116 clean and dry the surface prior to application of the material improving adhesion of the material to the surface. This is particularly important for surfaces in the mouth, such as a tooth surface, that due to the conditions within the mouth and on the tooth surface often prevent materials from adhering.

FIG. 5C is a slightly modified embodiment having absorbent post 116′ that has a rounded distal end. The rounded distal end may provide improved comfort for a user.

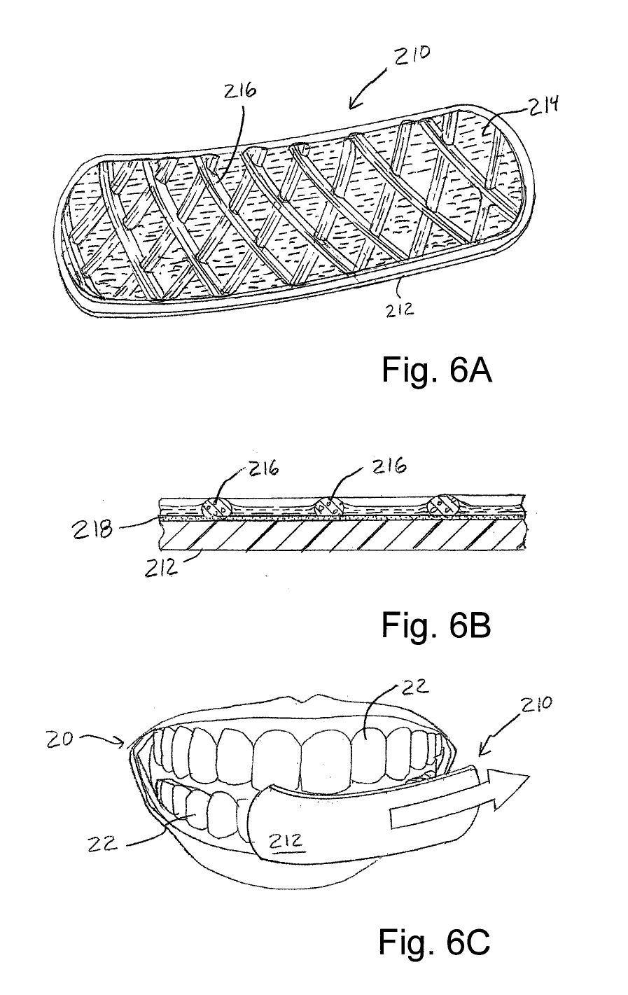

FIGS. 6A-C illustrate another embodiment of the present invention having a raised grid 216. The cleaning, drying, and dispensing applicator pad 210 has a flexible substrate 212 with a material 214 thereon. Placed on top of the substrate 212 is a raised grid 216 having the material 214 interspersed among the openings within the raised grid 216. An adhesive layer 218 may be applied between the material layer 214 and the substrate 212 to aid in maintaining the material 214 in place. FIG. 6C illustrates the operation of the pad 210 in cleaning, drying, and applying a material to teeth 20 in a person's mouth 20. The absorbent post illustrated in FIGS. 5A-C may also be configured or aligned to comprise a grid structure.

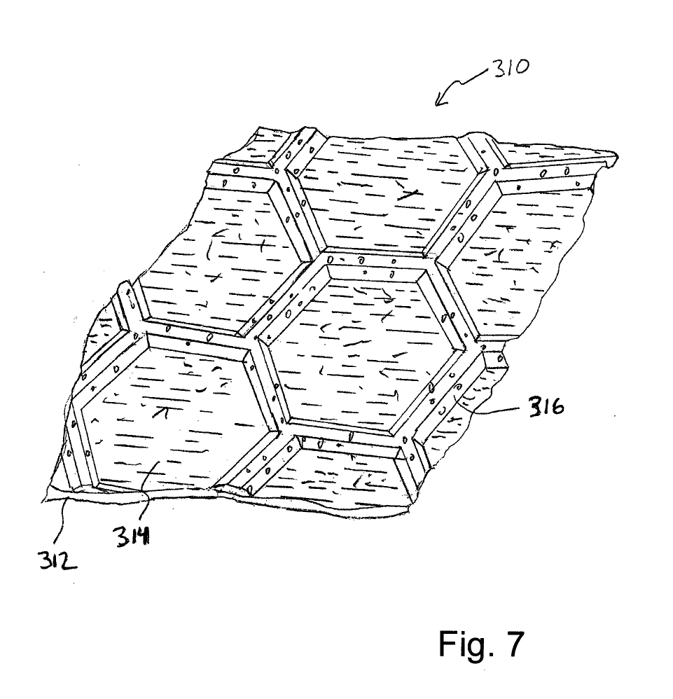

FIG. 7 illustrates another embodiment of a cleaning, drying, and dispensing applicator pad 310 of the present invention. In this embodiment, an absorbent geometric grid 316 is placed on the substrate 312 with the material 314 placed within the openings in the absorbent geometric grid 316. In this example the geometric grid forms a hexagon.

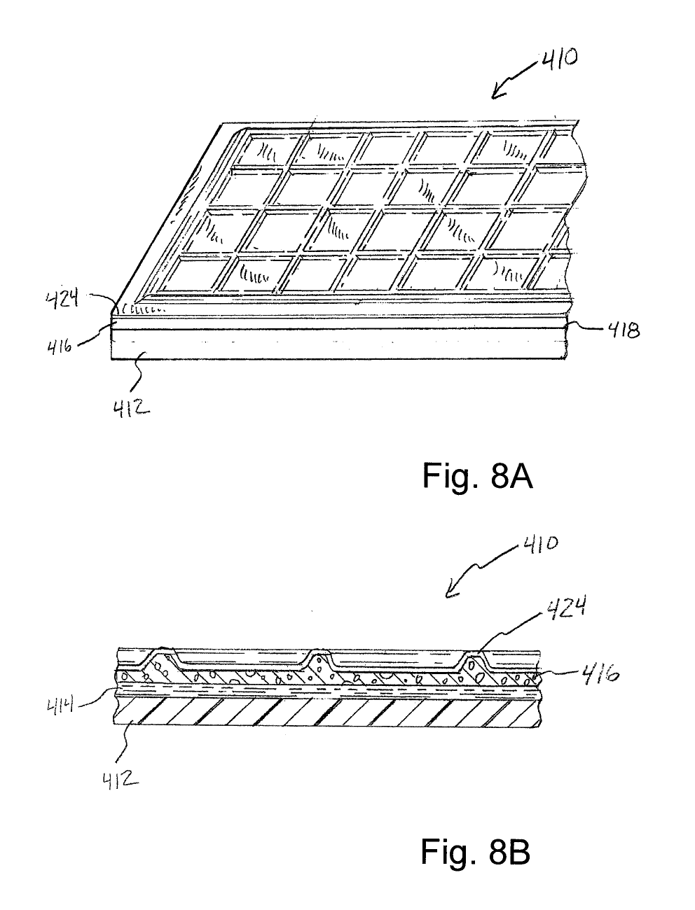

FIGS. 8A-B illustrate another embodiment of a cleaning, drying, and dispensing applicator pad 410 of the present invention. In this embodiment a medicated or medicinal material layer or material coating 424 is applied to the surface of a transmissible cover 416. The substrate 412 having the material 414 placed thereon is covered by the transmissible cover 416. The medicated carrier layer or material coating 424 placed on the transmissible cover 416 may be a cleaning, bonding, anti-fungal, or anti-bacteria agent. The material coating 424 therefore may be used to aid in the adherence of the material 414 when applied to the tooth or to kill germs and prevent bacteria growth. A seal 418 formed on the perimeter of the applicator pad 410 seals the material 414 therein. The seal 418 may be formed by an adhesive, pressure, heat or other means. A grid pattern on the transmissible cover is illustrated, but is not necessary.

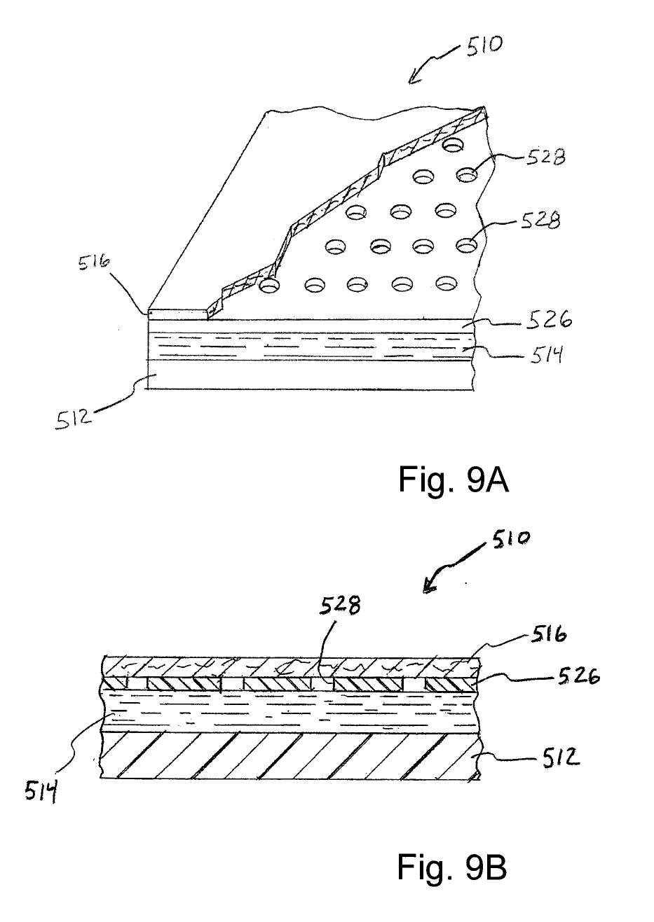

FIGS. 9A-B illustrate another embodiment of a cleaning, drying, and dispensing applicator pad 510 of the invention. In this embodiment a barrier film 526 having holes 528 therein is placed between a transmissible cover 516 and the material 514. The material 514 is placed on the substrate 512. In this embodiment the holes 528 better regulate the flow of the material 514 onto and through the transmissible cover 516. By appropriately sizing the holes 528, transfer of the material 514 to the transmissible cover 516 may be controlled or prevented until a slight pressure is applied to the substrate 512. Adhesive or glue may be used for adhering the transmissible cover 516 to the barrier film 26.

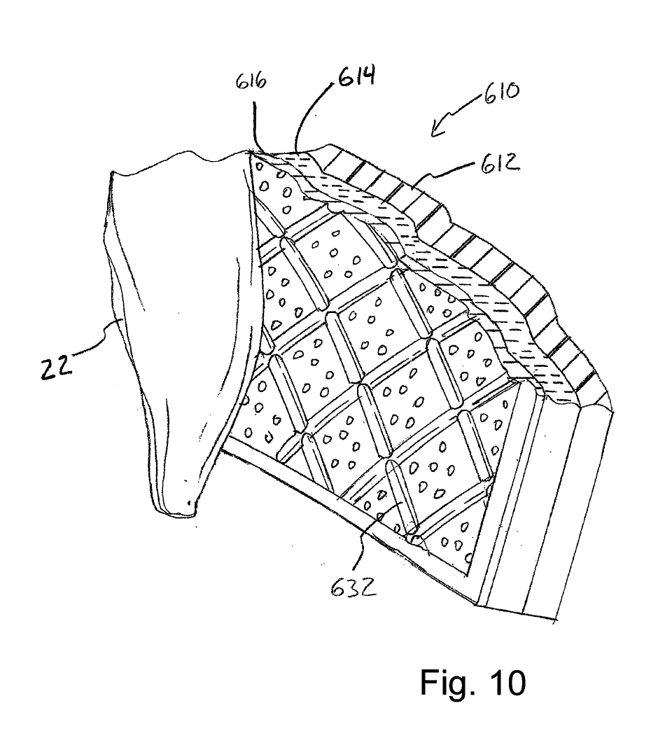

FIG. 10 illustrates another embodiment of a cleaning, drying, and dispensing applicator pad 610. In this embodiment a water repelling grid 632 is placed on top of a transmissible cover 616. A material 614 to be applied to the tooth 22 surface is placed between a flexible substrate 612 and the transmissible cover 616. The water repelling grid 632 aids in cleaning the surface of a tooth 22 as well as pushing moisture off of the surface of the tooth 22 preparing it for receiving the material 614. The material 614 is transmitted through the transmissible cover 614 upon the application of pressure to the substrate 612. This provides a more effective cleaning of the tooth 22 surface.

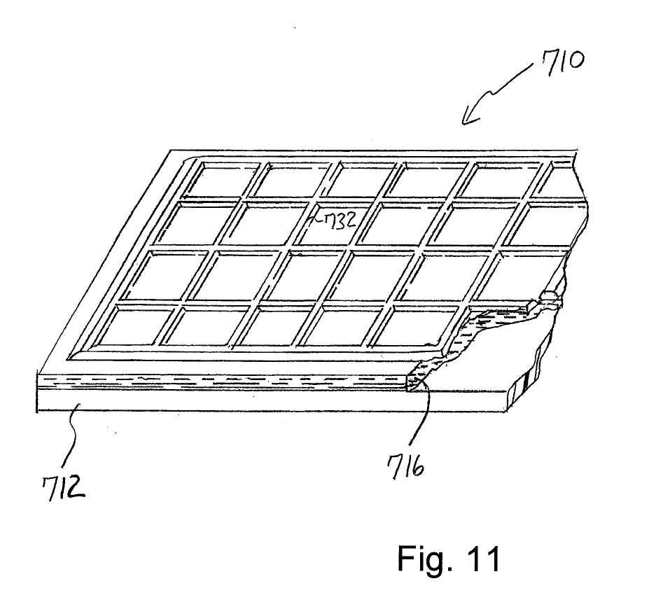

FIG. 11 illustrates another embodiment of a clean, drying, and dispensing applicator pad 710 of the present invention. In this embodiment an impregnated top layer 716 is placed on the flexible substrate 712. The impregnated top layer 716 may be made of any material capable of absorbing a material, such as cotton, cellulose, or other porous. The impregnated top layer 716 may be impregnated with a material to be applied to a tooth surface. A grid 732 is illustrated, but is not necessary.

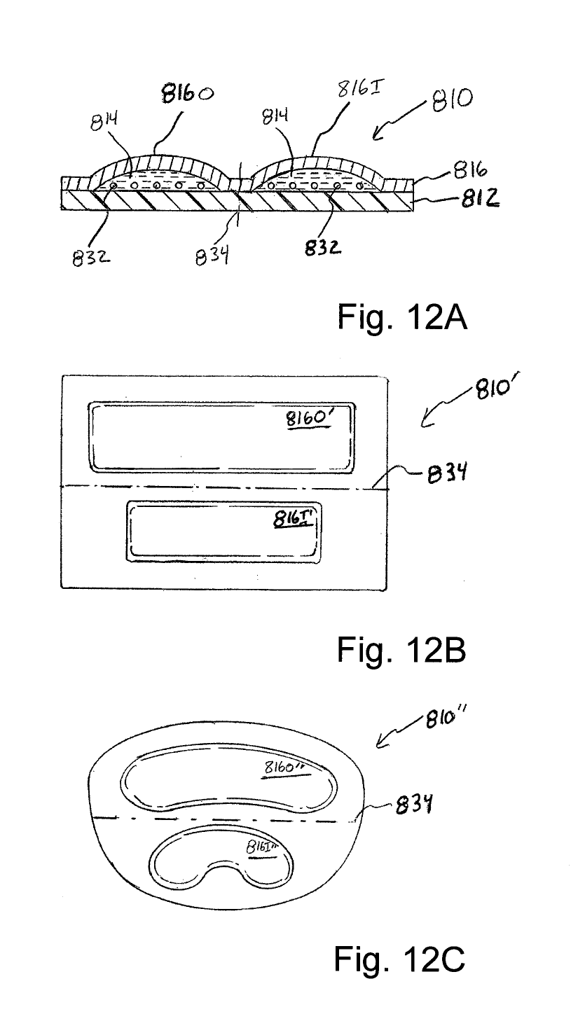

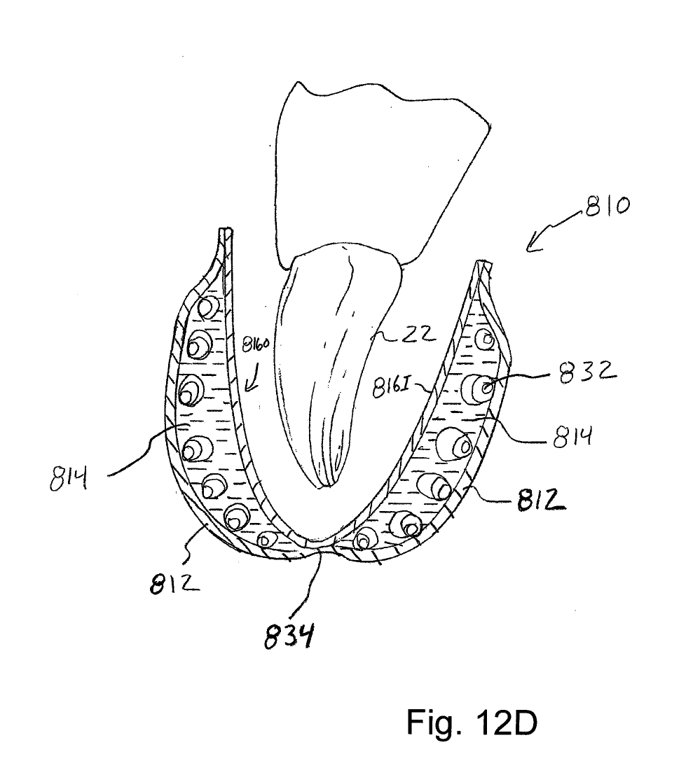

FIGS. 12A-D illustrate other embodiments of the invention. These embodiments relate to a dual applicator pad 810, 810′, and 810″. In FIG. 12A a cross-section of a dual applicator pad 810 is illustrated. The dual applicator pad 810 comprises a flexible and bendable substrate 812 with a wax material 814 held within a mesh structure 832 and covered by a transmissible cover 816. The mesh structure 832 provides some rigidity or strength to a portion of the dual applicator pad 810 facilitating folding along fold line 834. The fold line 834 divides the dual applicator pad 810 into two portions or sections; an outer tooth pad 8160 and inner tooth pad 816I.

FIG. 12B illustrates an embodiment of a dual applicator pad 810′ having different size and generally rectangular shaped pads, an outer tooth pad 8160′ and inner tooth 816I′ separated by a fold line 834.

FIG. 12C illustrates another embodiment of a dual applicator pad 810″ having different size curved or anatomically shaped pads; an outer tooth pad 8160″ and an inner tooth pad 816I″. The anatomically shaped pads are shaped to better conform to a tooth surface when folded and positioned in the mouth for application of the material to the tooth surface.

FIG. 12D illustrates the use of the embodiments of the invention illustrated in FIGS. 12A-C. When the dual applicator pad 810 is folded about the fold line 834 the outer tooth pad 816O is positioned adjacent an outer surface of the tooth 22 and the inner tooth pad 816I is positioned adjacent the inner surface of the tooth 22. This embodiment permits simultaneous application of the material 814 to the outer and inner surfaces of the tooth 22. After the dual applicator pad 810 is folded around the tooth 22 the outer tooth pad 816O and inner tooth pad 816I may be gently pressed by two fingers to dispense or apply the material 814 through the transmissible cover 816 onto the inner and outer surfaces of the tooth 22. In some applications, a different material may be placed in each outer tooth pad 816O and inner tooth pad 816I.

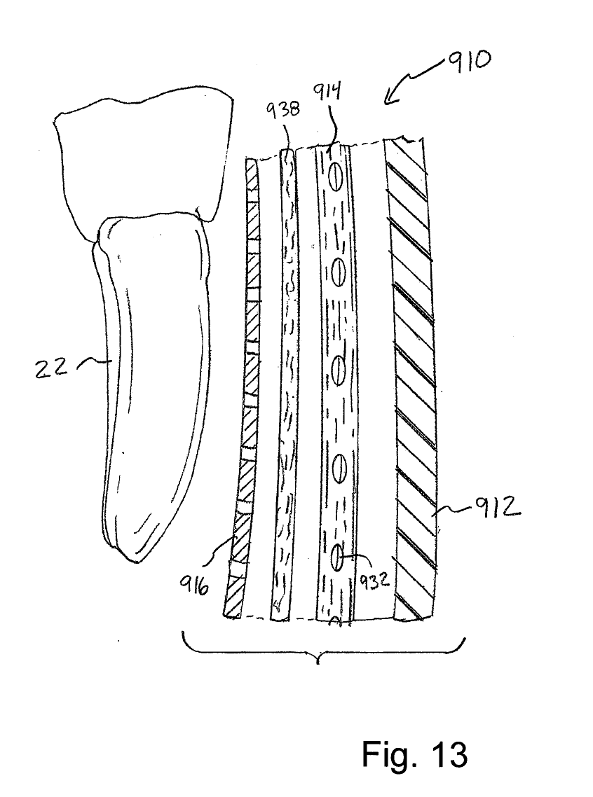

FIG. 13 schematically illustrates another embodiment of the invention. In this embodiment a medicament is additionally applied. The medicated applicator pad 910 comprises a transmissible cover 916 adjacent a medicated carrier layer 938 adjacent the material 914 held within a mesh 932 and placed on a flexible substrate 912. When the transmissible cover 916 is placed adjacent the surface of a tooth 22 the medicament on the medicated carrier layer 938 is caused to pass through the transmissible cover 916. The medicated applicator pad 910 may then be gently squeezed to apply the material 914. Alternately, the application can occur in a single step by gently squeezing the medicated applicator pad 910 causing the material 914 to pass through the medicated carrier layer 938 picking up the medicament and then pass through the transmissible cover 916 for simultaneous placement on the surface of the tooth 22. The transmissible cover 916 may be a water absorption layer and can be made of a cotton cloth. The medicated carrier layer 938 may contain any material such as a primer to treat a tooth surface for better bonding, medicine to kill germs, bleaching agents, fluoride varnish reagents, or materials to alter the hydrophobicity of a tooth surface. Another transmissible layer, not shown, can be inserted between medicated carrier layer 938 and material 814 in order to maintain separation of material 914 and medicated carrier layer 938.

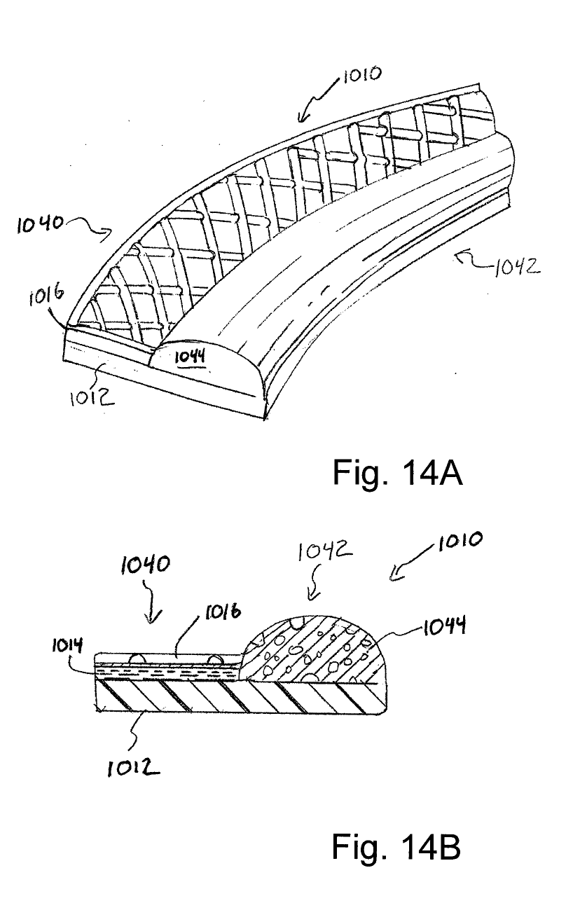

FIGS. 14A-B illustrate yet another embodiment of the invention. In this embodiment a combination applicator pad 1010 comprises a material applicator portion 1040 and a drying portion 1042. The material applicator portion 1040 comprises a transmissible cover 1016 adjacent a material 1014 placed on a substrate 1012. A grid may be placed on the transmissible cover 1016, but is not necessary. The drying portion 1042 comprises an absorbent material 1044 placed on the substrate 1012. The absorbent material 1044 may be any material that readily absorbs moisture or fluid.

Accordingly, the present invention provides an applicator pad that greatly facilitates and improves the application of a material to the surface of a tooth permitting the material to adhere to the tooth surface for a longer period of time. This is practically advantageous when used in combination with a wax or other protective material to prevent staining of a tooth between whitening treatments. The invention is also applicable to applying a tooth whitening agent and fluoride treatment. The invention may also be applied to other materials used for coating a wet and/or moist surface, such as a tooth.

What is claimed is:

1. A pre-dosed cleaning, drying, and dispensing pad comprising:
a flexible impervious substrate, said flexible imperious substrate adapted to conform to an irregular surface;
a wax material to be dispensed on the irregular surface placed uniformly on said flexible impervious substrate; and
an absorbent transmissible cover placed over said wax material and attached to said flexible impervious substrate,
whereby the irregular surface is cleaned and dried when wiped with said absorbent transmissible cover and a coating of said wax material is applied to the irregular surface.

2. A pre-dosed cleaning, drying, and dispensing pad as in claim 1 further comprising:
a barrier film having holes therein placed between said wax material and said absorbent transmissible cover.

3. A pre-dosed cleaning, drying, and dispensing pad as in claim 1 further comprising:
a raised grid placed on said absorbent transmissible cover.

4. A pre-dosed cleaning, drying, and dispensing pad as in claim 3 wherein:
said raised grid comprises a water repellent material.

5. A pre-dosed cleaning, drying, and dispensing pad as in claim 1 wherein:
said wax material comprises a tooth whitening agent.

6. A pre-dosed cleaning, drying, and dispensing pad as in claim 1 wherein:
said wax material comprises a fluoride material.

7. A pre-dosed cleaning, drying, and dispensing pad as in claim 1 further comprising:
a fold line, wherein the cleaning, drying, and dispensing pad is divided into at least two pad portions.

8. A pre-dosed cleaning, drying, and dispensing pad as in claim 1 further comprising:
a medicated carrier layer placed adjacent said absorbent transmissible cover.

9. A pre-dosed cleaning, drying, and dispensing applicator pad for applying a material comprising:
a flexible substrate, said flexible substrate adapted to conform to an irregular surface;
a plurality of flexible absorbent posts having distal ends formed on said flexible substrate, said plurality of absorbent posts extending from said substrate whereby each of said plurality of flexible absorbent posts bend when contacting the irregular surface; and
a wax material to be dispensed on the irregular surface placed uniformly on said flexible substrate and between said plurality of flexible absorbent posts, said wax material having a top surface between said flexible substrate and the distal ends of said plurality of flexible absorbent posts,
whereby when said plurality of flexible absorbent posts contacts the irregular surface, the distal ends absorb liquid on the irregular surface and bend permitting application of said wax material evenly on the irregular surface promoting adhesion of the wax material to the irregular surface.

10. A pre-dosed cleaning, drying, and dispensing applicator pad for applying a material as in claim 9 wherein:
said wax comprises a tooth whitening agent.

11. A pre-dosed cleaning, drying, and dispensing applicator pad for applying a material as in claim 9 wherein:
said wax comprises a fluoride material.

12. A pre-dosed cleaning, drying, and dispensing applicator pad for applying a material comprising:
a flexible substrate, said flexible substrate adapted to conform to an irregular surface;
a flexible absorbent grid having openings formed on said flexible substrate; and
a wax material to be applied to the irregular surface placed uniformly in the openings of said flexible absorbent grid, said wax material having a top surface between said flexible substrate and a top surface of said flexible absorbent grid,
whereby when said flexible grid contacts the irregular surface, the grid cleans and dries the irregular surface and compresses permitting application of said wax material evenly on the irregular surface promoting adhesion of said wax material to the irregular surface.

13. A pre-dosed cleaning, drying, and dispensing applicator pad for applying a material as in claim 12 further comprising:
adhesive placed between said flexible substrate and said flexible absorbent grid and said wax material

14. A pre-dosed cleaning, drying, and dispensing applicator pad for applying a material as in claim 12 wherein:
said flexible grid has a perimeter forming a geometric shape.

15. A pre-dosed cleaning, drying, and dispensing applicator pad for applying a material to opposing outer and inner surfaces comprising:
a flexible impervious substrate;
a first wax material to be dispensed on the outer surface placed on a first portion of said flexible impervious substrate;
a second wax material to be dispensed on the inner surface placed on a second portion of said flexible impervious substrate;
an absorbent transmissible cover placed over said first and second wax materials and attached to said flexible impervious substrate around a first perimeter of said first wax material and a second perimeter of said second wax material, whereby said first and second wax materials are separated from each other; and
a fold line formed in said flexible impervious substrate between said first and second wax materials,
whereby the opposing outer and inner surfaces are cleaned and dried when wiped with said absorbent transmissible cover and a coating of said first wax material is applied to the outer surface and a coating of said second wax material is applied to the inner surface.

16. A pre-dosed cleaning, drying, and dispensing applicator pad for applying a material to opposing outer and inner surfaces as in claim 15 wherein:
the first and second perimeters form a curved anatomical shape.

17. A pre-dosed cleaning, drying, and dispensing applicator pad for applying a material to opposing outer and inner surfaces as in claim 15 wherein:
said wax material comprises a tooth whitening agent.

18. A pre-dosed cleaning, drying, and dispensing pad comprising:
a flexible substrate, said flexible substrate adapted to conform to an irregular surface and having opposing longitudinal edges;
an absorbent material attached to a portion of said flexible substrate along one of said opposing longitudinal edges;
a wax material placed on a remaining portion of said flexible substrate along another one of said opposing longitudinal edges; and
an absorbent transmissible cover placed over said wax material,
whereby said absorbent material is capable of cleaning and drying the irregular surface prior to applying said wax material to the irregular surface.

19. A pre-dosed cleaning, drying, and dispensing pad as in claim 18 wherein:
said wax material comprises a tooth whitening agent.

20. A pre-dosed cleaning, drying, and dispensing pad as in claim 18 wherein:
said wax material comprises a fluoride material.

21. A method of applying a material to a tooth surface comprising the steps of:
holding the material between a transmissible cover and an impervious substrate;
rubbing the tooth with the transmissible cover, whereby the tooth surface is dried;
applying pressure to the substrate sufficient to dispense the material through the transmissible cover,
whereby the transmissible cover cleans and dries the tooth surface and upon the application of pressure on said substrate the material passes through the transmissible cover coating the tooth surface with the material.

22. A method of applying a material to a tooth surface as in claim 21 wherein:
the material comprises a wax.

23. A method of applying a material to a tooth surface as in claim 21 wherein:
the wax comprises a tooth whitening agent.

24. A method of applying a material to a tooth surface as in claim 21 wherein:
the wax comprises a fluoride material.
